(19) United States
(12) Patent Application Publication
(10) Pub. No.: US 2019/0006038 A1
(43) Pub. Date: Jan. 3, 2019

(54) ADAPTIVE THERAPY MANAGEMENT SYSTEM

(71) Applicant: Wendy Hajik, Addison, TX (US)

(72) Inventor: Wendy Hajik, Addison, TX (US)

(21) Appl. No.: 16/022,992

(22) Filed: Jun. 29, 2018

Related U.S. Application Data
(60) Provisional application No. 62/527,405, filed on Jun. 30, 2017.

(51) Int. Cl. G16H 20/30; A61B 5/00

(52) U.S. Cl. CPC G16H 20/30; A61B 5/4836; A61B 5/4824

(57) ABSTRACT

An adaptive therapy management system enables those skilled in message therapy and conventional treatments to select and apply known massage techniques, in combination with exercise, stretches, and movement patterns, to eliminate tissue pain by correcting the cause of the pain. A database contains known massage techniques, exercise, and stretches and movement patterns, each having associated application criteria whereupon they will properly eliminate the source of pain. The techniques being grouped in protocols for application.

### BACKGROUND

### 1. Field of the Invention

The present invention relates generally to soft tissue therapy methods, and more specifically, to an adaptive therapy management system for modifying the treatment and care of a subject dependent on the specific presented conditions.

### 2. Description of Related Art

Soft tissue therapy methods are well known in the art and are effective means to treat soft tissue ailments or injuries through non-medicinal means. For example, FIG. 1 depicts a conventional massage therapy session 101 having a series of preselected techniques 103, 105 and 107 that are performed regardless of the condition of the subject. During use, a therapist will apply the techniques 103 to the subject.

One of the problems commonly associated with system 101 is its limited efficiency. For example, a subject could present one of various conditions for treatment and the preselected techniques may or may not address the specific condition of the subject. While the preselected techniques are intended to cover a majority of the possible conditions of any subject, they do not focus on the needs of the individual subjects.

Accordingly, although great strides have been made in the area of conventional massage therapy sessions, many shortcomings remain.

### DESCRIPTION OF THE DRAWINGS

The novel features believed characteristic of the embodiments of the present application are set forth in the appended claims. However, the embodiments themselves, as well as a preferred mode of use, and further objectives and advantages thereof, will best be understood by reference to the following detailed description when read in conjunction with the accompanying drawings, wherein:

FIG. 1 is a flowchart of a common massage therapy session;

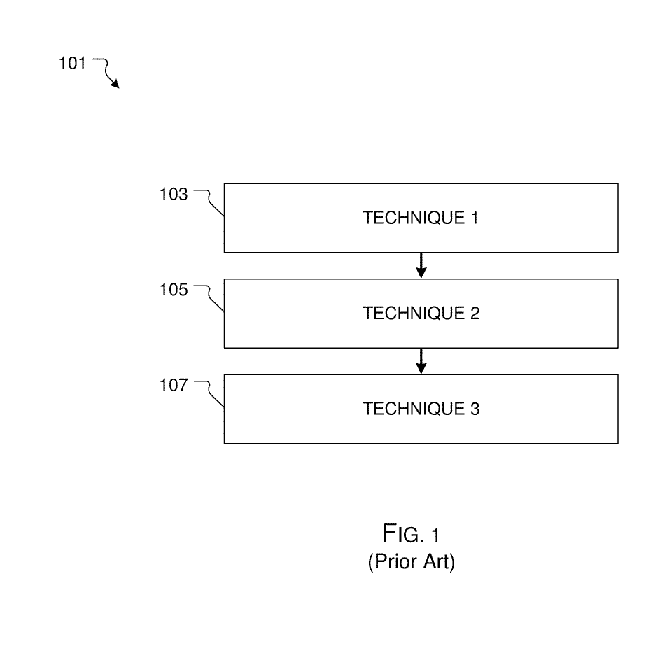

FIG. 2 is a diagram of an adaptive therapy management system in accordance with a preferred embodiment of the present application;

FIG. 3 is a flowchart of the preferred method of use of the system of FIG. 2; and

FIG. 4 is a diagram of a protocol of the system of FIG. 2.

While the system and method of use of the present application is susceptible to various modifications and alternative forms, specific embodiments thereof have been shown by way of example in the drawings and are herein described in detail. It should be understood, however, that the description herein of specific embodiments is not intended to limit the invention to the particular embodiment disclosed, but on the contrary, the intention is to cover all modifications, equivalents, and alternatives falling within the spirit and scope of the present application as defined by the appended claims.

### DETAILED DESCRIPTION OF THE PREFERRED EMBODIMENT

Illustrative embodiments of the system and method of use of the present application are provided below. It will of course be appreciated that in the development of any actual embodiment, numerous implementation-specific decisions will be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The system and method of use in accordance with the present application overcomes one or more of the above-discussed problems commonly associated with conventional massage therapy session. Specifically, the system of the present application enables the modification of techniques to be administered to a subject dependent on the presented conditions. These and other unique features of the system and method of use are discussed below and illustrated in the accompanying drawings.

The system and method of use will be understood, both as to its structure and operation, from the accompanying drawings, taken in conjunction with the accompanying description. Several embodiments of the system are presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless described otherwise.

The preferred embodiment herein described is not intended to be exhaustive or to limit the invention to the precise form disclosed. It is chosen and described to explain the principles of the invention and its application and practical use to enable others skilled in the art to follow its teachings.

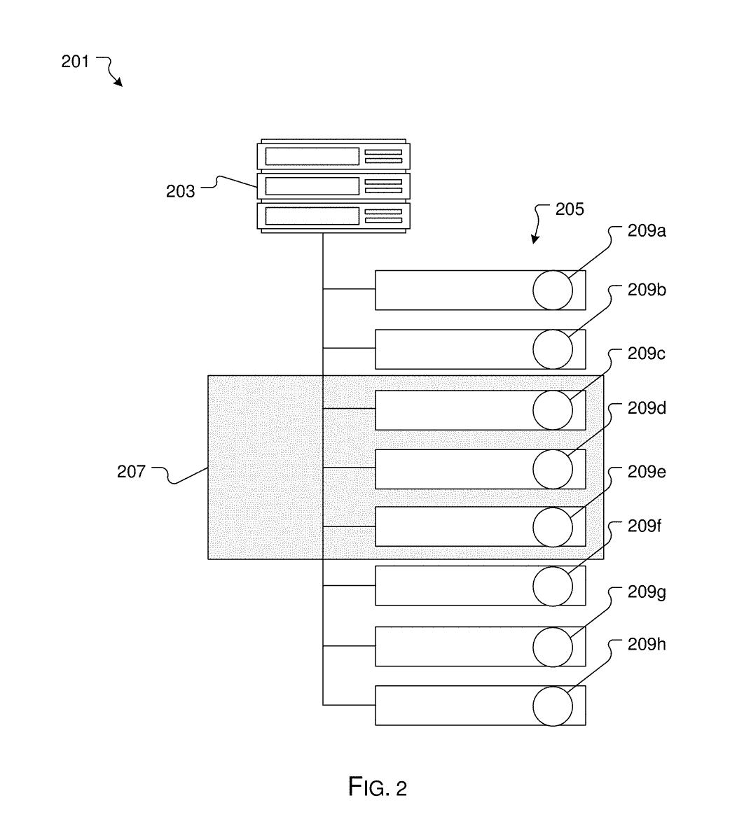

Referring now to the drawings wherein like reference characters identify corresponding or similar elements throughout the several views, FIG. 2 depicts a diagram of an adaptive therapy management system in accordance with a preferred embodiment of the present application. It will be appreciated that system 201 overcomes one or more of the above-listed problems commonly associated with conventional massage therapy sessions.

In the contemplated embodiment, system 201 includes a database 203 of potential techniques 205 to be selected and grouped in protocols 207 as part of a treatment 209. Each technique 205 having associated criteria 211 for their application and use.

It should be appreciated that one of the unique features believed characteristic of the present application is that database 203 with its techniques 205 are available to be grouped in protocols 207 dependent of the presentation of the subject. It will further be appreciated that various levels of treatment are selected and proscribed dependent on the needs of the subject and the presented pain. It will be understood that the care of each subject can be accommodated and administered through the use of the technique 205 database 203 and that updates to the techniques are anticipated.

It will be appreciated that the techniques 205 are any type of movement or interaction such as but not limited to, soft tissue manipulation, tool application, corrective exercise with and without restraint, stretches or movement patterns. It will also be appreciated that the techniques 205 could be taught and then performed by the subject at a later time.

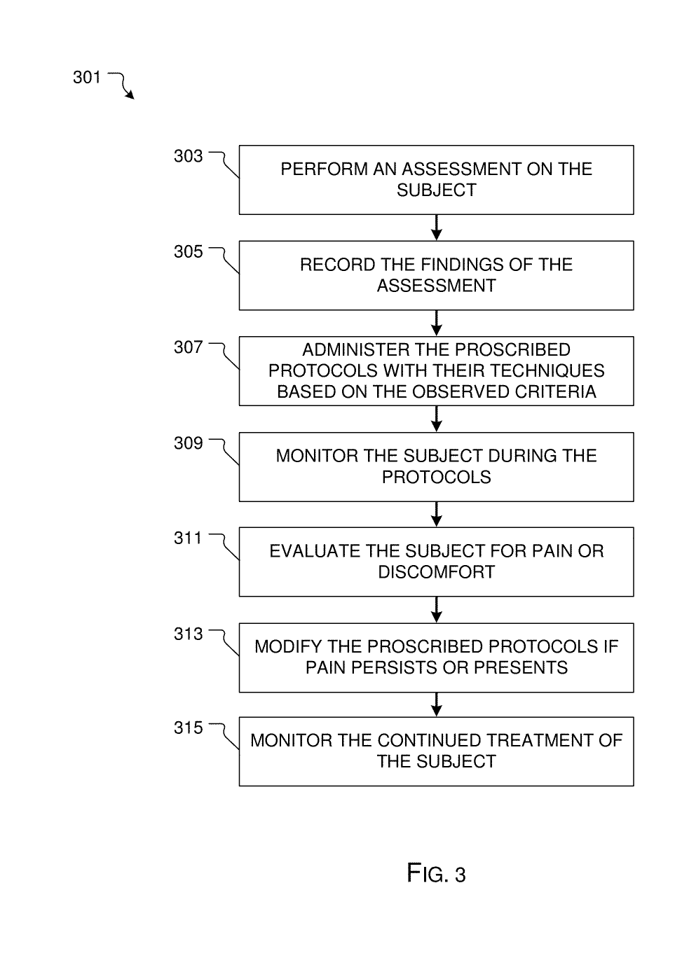

Referring now to FIG. 3 the preferred method of use of the system 201 is depicted. Method 301 including performing an assessment on the subject 303, recording the findings of the assessment 305, administering the proscribed protocols with their techniques based on the observed criteria 307, monitoring the subject during the protocols 309, evaluating the subject for pain or discomfort 311, modifying or expanding the proscribed protocols if needed 313 and monitoring the continued treatment of the subject 315.

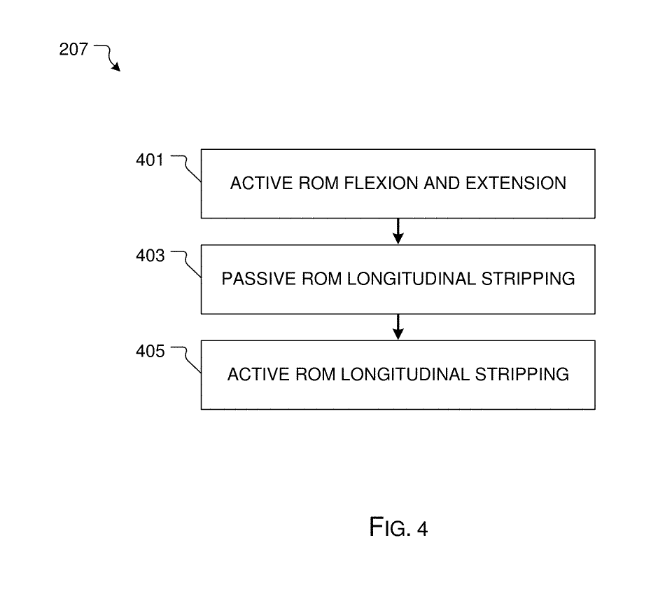

Referring now to FIG. 4 a protocol 207 of system 201 is depicted. Protocol 207 including techniques 401, 403 and 405. It will be understood that the techniques combine to treat the tissues at the root of the pain or discomfort experienced by the subject. It will be appreciated that by enabling techniques 401, 403, 405 to be combined to from protocol 207 by those not skilled in the art greatly expands the tools available to facilitate healing in the subjects. It will be understood that the intent of this disclosure is not to displace or remove conventional certifications and licensing common in the art, but to provide an additional tool.

The particular embodiments disclosed above are illustrative only, as the embodiments may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. Although the present embodiments are shown above, they are not limited to just these embodiments, but are amenable to various changes and modifications without departing from the spirit thereof.

What is claimed:

1. An adaptive therapy management system comprising:
a database of techniques to be selected and grouped in protocols;
each technique having criteria for selection;
wherein those not skilled in the art are able to select and administer techniques to eliminate tissue pain.

2. A method of creating and administering therapy given the system of claim 1, comprising:
performing an assessment on the subject;
recording the findings of the assessment;
administering the proscribed protocols with their techniques based on the observed criteria;
monitoring the subject during the protocols;
evaluating the subject for pain or discomfort;
modifying or expanding the proscribed protocols if needed; and
monitoring the continued treatment of the subject.
